(19) United States
(12) Patent Application Publication
(10) Pub. No.: US 2007/0272281 A1
(43) Pub. Date: Nov. 29, 2007

(54) Aerosol-Deliverable Formulations

(75) Inventor: Peiter Axel Pel, Delft (NL)

Correspondence Address: MORGAN LEWIS & BOCKIUS LLP, 1111 PENNSYLVANIA AVENUE NW, WASHINGTON, DC, 20004, US

(73) Assignee: QUEST INTERNATIONAL SERVICES, NL-1411 GP Naarden (NL)

(21) Appl. No.: 11/663,627

(22) PCT Filed: Sep. 21, 2005
(86) PCT No.: PCT/IB05/03109

§ 371 Date: Mar. 21, 2007

(30) Foreign Application Priority Data
Sep. 24, 2004 (GB) 0421291.6

(51) Int. Cl. C11D 17/00; A61K 8/04; C11D 3/12; C11D 3/50

(52) U.S. Cl. 134/34; 510/397

(57) ABSTRACT

An aerosol-deliverable formulation comprises a liquid abrasive composition having a viscosity of 1000 mPas or less, and a propellant. The formulation provides effective, and unexpected, suspensions of abrasives that do not settle from the suspension to form a hard cake of non-redispersible solid material.

### FIELD OF THE INVENTION

This invention relates to aerosol-deliverable formulations, i.e. formulations that can be delivered in the form of an aerosol spray, foam or mousse from a pressurised container containing a propellant gas under pressure. The invention concerns aerosol-deliverable formulations comprising liquid abrasive compositions.

### BACKGROUND TO THE INVENTION

Liquid abrasive compositions, particularly liquid abrasive cleaning compositions such as household cleaners, are well known and can be very effective in cleaning from hard surfaces tough stubborn soils, greases, burnt-in materials and stains which are often not completely removed by abrasive-free products.

Typically, liquid abrasive cleaning compositions comprise suspensions of small particles of abrasive material, such as calcite, silica, magnesium silicate or the like in an aqueous phase comprising water, with optional surfactants and/or polymers to provide physical stability and surfactancy properties.

Many publications deal with the formulation of liquid abrasive cleaning compositions. For example, U.S. Pat. No. 5,679,877 shows an example of a thickened liquid cleaning composition containing an abrasive; U.S. Pat. No. 5,286,405 refers to polymer-thickened liquid abrasive cleaning compositions; U.S. Pat. No. 4,911,857 describes an aqueous liquid abrasive cleaning composition with particulate abrasive suspended in aqueous medium; U.S. Pat. No. 4,842,757 relates to thickened liquid abrasive cleanser with improved stability; U.S. Pat. No. 4,840,746 discloses a liquid cleanser composition containing an abrasive crystalline aluminosilicate zeolite aggregate; and U.S. Pat. No. 4,751,016, U.S. Pat. No. 4,302,347, U.S. Pat. No. 4,284,533 and U.S. Pat. No. 3,997,460 all disclose liquid abrasive cleaners.

Disadvantages associated with current liquid abrasive cleaning compositions include the fact that they are not easily applied to and will not cling to vertical surfaces. Further, they are not perceived to work without intensive rubbing/scouring and, as a result, they are associated by the consumer with a requirement for hard work. In addition, current liquid abrasive cleaning compositions show limited fragrance impact due to their being used undiluted at room temperature and on relatively small surface areas.

Aerosol cleaning foams/mousses are well known and used, for example, for carpet cleaning and bathroom cleaning. These products appeal to consumers as they are convenient, they can be applied with one hand and there is no requirement to make a pre-dilution in water. Additionally, there is a perception amongst consumers that foams or mousses “work on dirt” once they have been sprayed on, without the need to apply heavy physical scouring. An additional appeal associated with foams/mousses is their strong fragrance impact due to their high specific surface area and the gradual collapsing of foam/mousse bubbles causing continuous release of trapped fragrance.

However, current aerosol cleaning foams/mousses are perceived as being not as effective as abrasive cleaners in removing tough dirt and, in general, they are viewed as being “light duty” cleaners

Typical aerosol foam products are described in PCT Patent Application No. WO02/097018 and in Canadian Pat. No. 994,634.

GB 945333 discloses a pourable liquid abrasive cleanser having a viscosity between 7,000 and 30,000 centipoise (or mPas) that may be dispensed from a pressure propelled dispenser such as an aerosol dispenser by the action of a propellant gas.

WO 2004/009049 discloses a thickened high viscosity dentifrice formulation including particulate abrasive, that is dispensable as a foam from a pressurised container by the action of a liquified propellant gas.

We have now surprisingly found that a relatively low viscosity liquid abrasive composition (unlike the compositions disclosed in GB 945333 and WO 2004/009049), can be delivered in the form of an aerosol. Hitherto, it was expected that the particles of abrasive material would settle out of the suspension and form a hard cake of solid material at the bottom of an aerosol container, in a process known as hard settling. It was also believed that, even if the abrasive material was successfully suspended in solution, it would block the aerosol stem, valve and/or actuator on spraying or prevent the valve from closing properly after spraying.

### SUMMARY OF THE INVENTION

In one aspect, the present invention provides an aerosol-deliverable formulation, comprising a liquid abrasive composition and having a viscosity of 1000 mPas or less, and a propellant.

Viscosity values given in this specification are measured at 20° C. unless otherwise specified.

Preferably the liquid abrasive composition is present in an amount in the range 30 to 95% by weight, more preferably 60 to 92% by weight, and the propellant is preferably present in an amount in the range 5 to 70% by weight, more preferably 8 to 40% by weight.

The present invention also provides an aerosol-deliverable composition comprising from 60 to 92% by weight of a liquid abrasive cleaning composition, preferably having a viscosity of 1000 mPas or less, and from 8 to 40% by weight of a propellant.

The liquid abrasive composition comprises particles of abrasive material suspended or suspendible in a liquid vehicle. While the particles may settle out of the liquid on storage, surprisingly the compositions in the formulations of the invention are found to be free from hard settling, so the particles do not form a hard cake of solid material. Instead the particles are readily redispersible into suspension with light shaking or agitation, to be in aerosol-deliverable form. It is surprising that this is possible with relatively low viscosity liquid abrasive compositions. The present invention thus enables aerosol delivery of liquid abrasive compositions that can be in the form of simple water-based compositions not requiring the use of thickeners (that add to manufacturing complexity, time and cost that may potentially lead to problems of valve blockage, and that may not be suited to production of a foam). It is also not necessary to use suspension aids, which might interfere with the functioning of the liquid abrasive composition, e.g. a cleaning function, and which might leave an undesirable film on a surface being treated. The present invention can thus provide simple and cheap yet effective formulations. Further, because the formulations are aerosol-deliverable, they can be delivered in the form of foams or mousses which tend to adhere to surfaces, even vertical surfaces without run-off.

The liquid abrasive composition and the propellant form an emulsion on shaking or agitation. In the emulsion, the abrasive material of the liquid abrasive composition is in suspension, as discussed above. The emulsion is either stable or easily redispersed with shaking or agitation to be in aerosol-deliverable form.

Propellants typically used, such as liquefied hydrocarbon gases to be discussed below, are generally very apolar solvents. When added to liquid abrasive compositions (that are typically water-based) and shaken to form an emulsion, it is likely that the emulsion will separate out after time. In this case the propellant will form a separate liquid phase on top of the liquid abrasive composition. Within the liquid abrasive compositions, the particles of abrasive material may settle out of suspension, as noted above. Nevertheless we have found that with formulations in accordance with the invention an emulsion can be readily reformed with light shaking, to be in aerosol-deliverable form, even after extended storage.

The formulations of the present invention may be made by mixing together liquid abrasive composition and propellant to form an emulsion that is either stable or easily redispersed with shaking and prevents hard settling of the abrasive.

The formulation is suitably housed in a pressurised container, such as an aerosol container, having a valve and an actuator. The formulations can be readily produced in a conventional manner. In a typical process, the liquid abrasive composition is put into the container. Air is then flushed out of the container with propellant gas. A valve is then attached, e.g. crimped, to the container. The required amount of propellant gas is then injected into the container through the valve. The container is then shaken to homogenise and emulsify the contents.

It is surprisingly found that the emulsion obtained may be dispensed from an aerosol device fitted with any suitable combination of valve and actuator, as may be selected by one skilled in the art, without blockage of the valve, actuator or stem of the aerosol device. In terms of spraying performance and valve performance, results that are at least acceptable have been obtained with a variety of different valves and actuators tested. So far, the best results have been obtained with a range of valves and actuators supplied by Deutsche Prazisions-Ventil Gmbh in Hattersh\dot{e}im, Germany. One preferred combination comprises an activator selected from Vulkan APSL 0.020 and Vulkan APSL 0.025 in combination with the valve specified as [Kegel: 2* 0.020 Nylon geringt, Dichtung: Neopren SH 74 S-90 PH-F 05-1200-XE, Feder: V2A draht 0.021 länge 0.280 P, Gehäuse nyl 4sch kro, Teller: alu gold/mico PH-F. on]. Another preferred combination comprises an actuator selected from Kosmos APSL 0.020 F-3MM and Kosmos APSL 0.025 F-3MM with the valve specified as [Kegel: 1* 0.024A puder schaft 3 mm ID1.5, Dichtung: Neopren SH74 PH-F 05-1250-XE, Feder: V2A draht 0.023 länge 0.340 P, Gehäuse 0.080 nyl kra stu 0.412 hub, Teller: alu gold mico puder PH-F on, Steigrohr STD-PE Inn 3.15, gesamtlänge 205 mm]. (Vulkan and Kosmos are Trade Marks).

The formulation of the present invention is suitably delivered as a mousse or foam. This enables the user to spray the formulation where desired and have the foam adhere to the surface without running. Where the composition includes fragrance, the foam also provides improved fragrance properties when compared with non-aerosol liquid abrasive compositions.

Suitable for use as the abrasive material in the liquid abrasive composition are both natural and synthetic mineral abrasives, for example dolomite, precipitated calcium carbonate (aragonite), feldspar, alumina, silica, abrasives such as quartz and quartzite; and preferably an abrasive material is used with a hardness on Moh's scale of from 1 to 4. Particularly suitable is calcite, for instance limestone, chalk or marble such as those forms of calcite referred to in the British Patent 1 345 119. Calcite has a density of 2.71 g/cc. In general, the average particle size of the abrasive material ranges from 1-70, usually 1-60, preferably 1-50 micrometers.

Preferably, the abrasive material will have a Mohs hardness of at least 2 and less than 3.

When the liquid abrasive compositions of this invention contain abrasives for hard surface cleaning, these will usually be incorporated as particulate solids. They may be those of the kind that are water insoluble, for example calcite. Suitable materials of this kind are disclosed in the European patent specifications EP-A-50,887; EP-A-80,221; EP-A-140,452; EP-A-214,540 and EP 9,942 (all assigned to Unilever PLC), which relate to such abrasives when suspended in aqueous media.

The abrasive material is typically at least substantially insoluble in water, but may be water insoluble or water-soluble. When a water-soluble abrasive is used, it preferably has a solubility in water of at least 5 g/l at 10° C., to ensure that any surplus abrasive material can readily be rinsed from a hard surface after cleaning with the formulation. In this way, the surface can be free from residual spots or streaks.

The total amount of abrasive material present in the liquid composition, both in dissolved and undissolved form, is suitably from 6 to 45%, preferably from 10 to 40%, and ideally 15 to 40% by weight. At least 5% by weight of the abrasive material should be present in the composition, at normal storage or use temperatures of from 10° C. to 40° C., in a solid, particulate form, having an average particulate size, and other characteristics, as herein specified.

The propellant may be any conventional aerosol propellant or combination of such propellants as are well known to those skilled in the art. For environmental reasons it is currently preferred that the propellant is a short chain hydrocarbon, such as propane, butane, isobutene, or mixtures thereof. Preferred propellants are mixtures of hydrocarbons formulated such that the pressure at 20° C. ranges between 2 and 8 bar.

The amount of propellant is preferably sufficient to provide full aerosol discharge. For reasons of economy, it is preferred not to use more propellant than necessary. Preferably, the propellant comprises from 5 to 70% by weight of the final product, more preferably from 8 to 40% by weight.

The liquid abrasive composition is desirably not in the form of an emulsion, and suitably lacks an oily phase. The composition is typically water-based.

The liquid abrasive composition is conveniently a liquid abrasive cleaning composition, preferably in the form of a household cleaner typically for cleaning hard surfaces such as floors, work surfaces, baths, showers, sinks etc.

The aerosol-deliverable liquid abrasive cleaning compositions of this invention is usually water-based, comprising water and optionally also other ingredients including surface active agents (surfactants), such as nonionic surfactants (including alcohol alkoxylates), anionic surfactants (including sulphates or sulphonates), cationic surfactants (including quaternary ammonium compounds) and amphoteric surfactants (including betaines); bleach ingredients, such as hypochlorite and hydrogen peroxide; polymers; enzymes; perfumes (including deoperfumes, malodour counteractant perfumes, insect repellent perfumes and/or mood enhancing perfumes); micro-biocides; colouring agents; fluorescers; corrosion inhibitors; and enzyme stabilizing agents.

We have discovered that fragrance compositions comprising at least one fragrance ingredient having an octanol-water partition coefficient (expressed as a logarithm to base 10) (i.e. log P) of 3 or more and a boiling point of 250° C. or lower, are particularly effective at providing a fragrance and freshening effect when used with the aerosol-dispensed liquid abrasive compositions of this invention.

Preferably, liquid abrasive composition comprises at least 30% by weight more preferably at least 40% by weight; and more preferably still at least 50% by weigh of at least one fragrance ingredient having a log P of 3 or more and a boiling point of 250° C. or lower.

The octanol-water partition coefficient (P) of a material i.e. the ratio of a material's equilibrium concentration in octanol and water, is well known in the literature as a measure of hydrophobicity and water solubility (see Hansch and Leo, Chemical Reviews, 526 to 616, (1971), 71; Hansch, Quinlan and Lawrence, J. Organic Chemistry, 347 to 350 (1968), 33). High partition coefficient values are more conveniently given in the form of their logarithm to the base 10, log P. While log P values can be measured experimentally i.e. directly, and measured log P data is available for many perfumes, log P values are most conveniently calculated or approximately estimated using mathematical algorithms. There are several recognised calculation or estimation methods available commercially and/or described in the literature (see for example A Leo, Chem. Rev 93(4), 1281-1306, (1993), “Calculating log P oct from structures”). Generally these models correlate highly but may for specific materials produce log P values which differ in absolute terms (by up to 0.5 log units or even more). However, no one model is universally accepted as the most accurate across all compounds. This is particularly true for estimates on materials of high log P (say 4 or greater). In the present specification, references to log P values means values obtained using the estimation software commercially available as ‘Log P’ from Toronto-based Advanced Chemistry Development Inc (ACD) which is well-known to the scientific community, and accepted as providing high-quality predictions of log P values.

Non-limiting examples of the more preferred fragrance ingredients are selected from the following, with even more preferred ingredients being identified by an *:
ALDEHYDE C10 (DECANAL)*
ALDEHYDE C11 (UNDECENAL)
ALDEHYDE C9 (NONANAL)
ALDEHYDE MNA
ALLYL AMYL GLYCOLATE*
ALLYL HEPTANOATE
ALLYL HEXANOATE
AMYL BUTYRATE
AZARBRE (Q)
BORNEOL
CARVACROL
CARYOPHYLLENE
CISTULATE (Q)
CITRAL
CITRAL DIETHYL ACETAL
CITRONELLAL
CITRONELLOL
CITRONELLYL ACETATE
CITRONELLYL NITRILE
PHENYLETHYL BUTYL ETHER
CUMIN NITRILE
CYCLOHEXYLETHYL ACETATE
CYMENE, para-
DAMASCENONE, beta-
DAMASCONE, alpha-
DAMASCONE, beta-
DAMASCONE, delta-
4-trans-DECENAL
DEC-9-ENOL
DIHYDROJASMONE
DIHYDROLINALOL
DIHYDROMYRCENOL*
DIHYDROMYRCENYL ACETATE
DIHYDROTERPINEOL (MENTHANOL)*
DIHYDROTERPINYL ACETATE*
ETHYL HEPTANOATE
ETHYL SAFRANATE
FLORHYDRAL
FRUTONILE (Q)
GERANIOL
GERANYL ACETATE*
GERANYL NITRILE*
HERBANATE
INONYL ACETATE*
ISOBORNYL ACETATE*
ISOPENTYRATE
JASMATONE (Q)
LIMONENE
LINALOL*
LINALYL ACETATE*
MACEAL (Q)
MAYOL
MENTHOL*
MENTHYL ACETATE
METHYL CHAVICOL
METHYL OCTINE CARBONATE
METHYL PAMPLEMOUSSE (G)
MYRCENE
NEO BERGAMATE FORTE
NEOCASPIRENE
NEROL
NOPYL ACETATE
OCTYL ACETATE
ORTHOLATE (Q)*
PARA TERT BUTYL CYCLOHEXANOL
PARA TERT BUTYL CYCLOHEXYL ACETATE*
PELARGENE (Q)
PHENETHYL ISOPROPYL ETHER
PHENYLETHYL ISOBUTYRATE
RHUBAFURAN (Q)
ROSE OXIDE
TERPINOLENE*
TERPINYL ACETATE*
TETRAHYDROGERANIOL
TETRAHYDROLINALOL
TETRAHYDROLINALYL ACETATE
TETRAHYDROMYRCENOL
TONALID
UNDECAVERTOL
VERTENEX
YLANGENE

Materials labelled “(Q)” are obtainable from Quest International, and those materials labelled “(G)” from Givaudan.

The aerosol-deliverable formulation of the invention can provide a combination of the benefits of liquid abrasive cleaners, such as excellent cleaning of tough dirt, with the convenience of use of an aerosol product, the ability to cling to vertical surfaces, the high fragrance impact and the “fun-factor” associated with aerosol cleaning foams/mousses.

The invention also provides a method of cleaning a surface, comprising the applying to the surface from a pressurised container a formulation in accordance with any one of the preceding claims, and removing surplus formulation after optional rubbing.

The formulation is typically applied as an aerosol spray, foam or mousse. After optional rubbing or wiping, surplus formulation is removed e.g. by wiping or rinsing off with water.

Although the present invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

The invention will further be described, by way of illustration, in the following non-limiting examples, in which quantities are expressed by percentages by weight of the total composition unless otherwise indicated.

### EXAMPLES

The following commercially available liquid abrasive cleaning compositions, were used in the examples:
Cif Cream (White variant), by Lever Fabergé, sold in Poland
Cif Cream Schuurmiddel Citroen (Yellow Variant), by Lever Fabergé, sold in the Netherlands
AH Schuurmiddel Citroen, by Albert Heijn, sold in the Netherlands

These cleaning compositions all comprise fine particles of calcium carbonate (10-50% w/w) dispensed in water with nonionic and/or anionic surfactants (1-10%) and perfume (0.1-1%) and possibly also polymers. The compositions are not in the form of emulsions, lacking an oily phase. The compositions all have a viscosity below 1000 mPas with viscosities being as follows: Cif Cream White: 776 mPas, Cif Cream Yellow: 732 mPas, Citroen 624 mPas, all measured at 20° C. using a Haake Rheostress 1 at 21 l/s for 2 minutes using the spindle Kegel D=60 mm/1° Ti.

### Example 1

Three 75 ml aluminium Lechner aerosol cans were filled in conventional manner with the formulations below and were fitted with D.P.V. 841 valves. In particular, the required amount of liquid abrasive cleaner was put into the aerosol can. Air was then flushed out of the can with the propellant gas. The valve was then crimped on the can. After that, the required amount of propellant gas was injected into the can through the valve. The can was shaken to homogenise and emulsify the contents.

The spraying effect was recorded using various commercially available aerosol actuators and details are given below.

| | Formulation | | |
|---|---|---|---|
| Ingredient | 1 | 2 | 3 |
| Cif Cream white | 30 g | 40 g | 50 g |
| Propellant | 25 ml | 15 ml | 5 ml |
| (prop/but 3.5 | (13.75 g) = | (8.25 g) = | (2.75 g) = |
| bar) | 31% w/w | 17% w/w | 5% w/w |
| Effect | Thin foam, | Thicker, more | Not enough |
| | rapidly | stable foam. Best | propellant to get |
| | collapsing | with shaving | the whole |
| | “running foam“ | cream actuator | contents out of |
| | effect. Best | | the can |
| | with standard | | |
| | actuator | | |

A “running foam” effect from Formulation 1 was shown when a fast sweep of the can was made over a surface whilst spraying. A crackling effect of the foam was heard whilst it collapsed after it has hit the surface, which appears to the user as a running effect. The “running foam” effect contributes to the “fun-factor” of the product.

Formulation 2 gave a nice, fine, creamy yet abrasive mousse, which showed very good ability to cling to vertical surfaces.

### Example 2

The following formulations were made up in aerosol cans as described in Example 1.

| | Formulation | |
|---|---|---|
| Ingredient | 1 | 2 |
| Cif Cream | 60 g | 80 g |
| white | | |
| Propellant | 50 ml | 30 ml |
| (prop/but | (27.5 g) = | (16.5 g) = |
| 3.5 bar) | 31% w/w | 17% w/w |
| Valve* | Kegel: 1*.024 A puder schaft | Kegel: 2* .020 Nylon |
| | 3 mm ID1.5, Dichtung: | geringt, Dichtung: |
| | Neopren SH74 PH-F 05-1250- | Neopren SH74 S-90 |
| | XE, Feder: V2A draht .023 | PH-F 05-12-XE, |
| | länge .340 P, Gehäuse .080 nyl | Feder: V2A draht .021 |
| | kra stu .412 hub, Teller: alu | länge .280 P, Gehäuse |
| | gold micro puder PH-F on, | nyl 4sch kro, Teller: |
| | Steigrohr STD-PE Inn 3.15, | alu gold/mico PH-F. |
| | gesamtlänge 205 mm. | on. |
| Actuator* | Kosmos APSL .025 F-3MM | Shaving cream |

*Valves and actuators were supplied by Deutsche Präzisions-Ventil GmbH in Hattersheim, Germany.

These formulations were stored at room temperature (20-25° C.) for over three months. Over these three months, the formulations were sprayed at irregular intervals after mild shaking. Valve blockage was not observed and the product sprayed out nicely as a creamy, yet abrasive mousse.

### Example 3

The formulations from Example 2 were stability tested in glass and tin aerosol cans at −10° C., 4° C., room temperature and 37° C. for 6, 8 and 12 weeks. During the evaluation points at 0, 6, 8 and 12 weeks, the cans were evaluated on various aspects, shaken to redisperse the calcite, sprayed and returned to storage. The following observations were made:

Appearance

Formulation 1 generally showed 4 layers in the glass aerosol can: from bottom to top classified as calcite sediment/cloudy/white/clear. Formulation 2 was nearly homogeneous at RT and 37° C. with a thin clear layer on top.

Dispersability

Formulation 1 generally needed serious shaking to redisperse the calcite in the formulation, whereas Formulation 2 (being more homogeneous) did not need shaking for the RT and 37° C. samples.

Weight Loss During Storage

No weight loss during storage was observed.

Valve/Actuator Blockage

With formulation 1, there were a few cases of valve or actuator blockage. With formulation 2, no valve or actuator blockage was observed

Mousse Appearance

Appearance of the “running foam” from Formulation 1 and the “creamy mousse” from. Formulation 2 remained constant over the test

Perfume Performance

Perfume performance from the mousses is excellent and remained constant in terms of strength and character.

### Example 4

In order to test the effect of different liquid abrasive compositions (bases) and different ratios of liquid abrasive composition and propellant, a range of samples was made in 100 ml glass aerosol containers with the aim of checking for sedimentation and redispersability.

The formulations used are given in the table below:

| | | | | Propellant |
|---|---|---|---|---|
| Formulation | Base | Propellant | Base [g] | [ml] |
| A | 91.6% | 8.4% | 60.00 | 10.00 |
| B | 82.9% | 17.1% | 50.91 | 19.09 |
| C | 75.7% | 24.3% | 44.21 | 25.79 |
| D | 68.6% | 31.4% | 38.18 | 31.82 |
| E | 60.9% | 39.1% | 32.31 | 37.69 |

The propellant used was a commercially available odourless propellant grade propane/butane/isobutene mixture, as supplied by Indugas, with a pressure of 3.5 Bar at 20° C., according to specification BS 4250.

The bases used were Cif Cream Schuurmiddel Citroen (Yellow Variant), by Lever Fabergé, sold in the Netherlands and AH Schuurmiddel Citroen, by Albert Heijn, sold in the Netherlands.

The formulations were made up in aerosol cans as described in Example 1.

The tables below show the presence of sediment and the effort required to redisperse the sediment that was present after storing the samples for three days at room temperature.

AH Schuurmiddel Citroen

| | | Effort to |
|---|---|---|
| Formulation | Sediment present | redisperse |
| A | Yes | Light shaking |
| B | Yes | Shaking |
| C | Yes | Light shaking |
| D | Yes | Tipping over |
| E | Yes | Light shaking |

Cif Cream Citroen

| | | Effort to |
|---|---|---|
| Formulation | Sediment present | redisperse |
| A | No | None |
| B | Yes | Light shaking |
| C | Yes | Shaking |
| D | Yes | Shaking |
| E | Yes | Shaking |

After storage for three months at 37° C., with all formulations the abrasive could be redispersed with shaking to light shaking.

Although the present invention has been described in considerable detail with reference to certain embodiments, one skilled in the art will appreciate that the present invention can be practised by other than the described embodiments, which have been presented for purposes of illustration and not of limitation. Therefore, the scope of the appended claims should not be limited to the description of the embodiments contained herein.

1. An aerosol-deliverable formulation, comprising a liquid abrasive composition having a viscosity of 1000 mPas or less, and a propellant.

2. A formulation according to claim 1, wherein the liquid abrasive composition is present in an amount in the range 30 to 95% by weight, preferably 60 to 92% by weight, and the propellant is present in an amount in the range 5 to 70% by weight, preferably .8 to 40% by weight.

3. A formulation according to claim 1 or 2, wherein the liquid abrasive composition comprises particles of abrasive material suspended or suspendible in a liquid vehicle.

4. A formulation according to claim 3, wherein the liquid abrasive composition comprises abrasive material, in undissolved and/or dissolved form, in an amount in the range 6 to 45% by weight, preferably 10-40% by weight, more preferably 15 to 40% by weight.

5. A formulation according to claim 4, wherein at least 5% of the abrasive material in the composition is in the form of undissolved particles at a temperature in the range 10 to 40° C.

6. A formulation according to claim 1 or 2, wherein the liquid abrasive composition and the propellant form an emulsion.

7. A formulation according to claim 1 or 2, wherein the liquid abrasive composition comprises a water-based non-emulsion.

8. A formulation according to claim 7, wherein the liquid abrasive composition comprises a liquid abrasive cleaning composition.

9. A formulation according to claim 8, wherein the liquid abrasive composition includes one or more fragrance materials.

10. A formulation according to claim 9, wherein the fragrance materials comprise at least one material having an octanol-water partition coefficient of at least 3 expressed as a logarithm to base 10, and a boiling point of 250° C. or less.

11. A formulation according to claim 1, in a pressurised container having a valve and actuator.

12. An aerosol-deliverable formulation comprising from 60 to 92% by weight of a liquid abrasive cleaning composition and from 8 to 40% by weight of a propellant.

13. A formulation according to claim 12, wherein the liquid abrasive cleaning composition has a viscosity of 1000 mPas or less.

14. A method of cleaning a surface, comprising the applying to the surface from a pressurised container a formulation in accordance with claim 1 or 2, and removing surplus formulation after optional rubbing.
